(19) United States
(12) Patent Application Publication
(10) Pub. No.: US 2025/0188646 A1
(43) Pub. Date: Jun. 12, 2025

(54) COMPOSITION TO BE ELECTROSPUN

(71) Applicant: BAKEL S.R.L., Udine (IT)

(72) Inventor: Raffaella GREGORIS, Udine (IT)

(21) Appl. No.: 18/844,848

(22) PCT Filed: Mar. 8, 2024
(86) PCT No.: PCT/IT2023/050065

§ 371 Date: Sep. 6, 2024

(30) Foreign Application Priority Data
Mar. 8, 2022 (IT) 102022000004418

(51) Int. Cl. D01D 1/02; A61L 15/28; A61L 15/32; A61L 15/48; D01D 5/00; D01D 5/253; D01F 1/02; D01F 9/00

(52) U.S. Cl. CPC D01D 1/02; A61L 15/28; A61L 15/32; A61L 15/325; A61L 15/48; D01D 5/003; D01D 5/253; D01F 1/02; D01F 9/00; D10B 2509/02

(57) ABSTRACT

The composition to be electrospun comprises a first compound to be electrospun and an electrospinning promoter, intended to stabilize the electrospinning of the first compound. It also describes a method for preparing the composition, which comprises mixing its components together in water.

### FIELD OF THE INVENTION

The present invention concerns a composition to be electrospun, i.e., allowing production of nanofibers by electrospinning. More particularly, the composition is of the type based on a polymer, favorably a biocompatible polymer, to be electrospun.

### BACKGROUND OF THE INVENTION

The electrospinning process that allows nanofibers, i.e., continuous fibers with a diameter of the order of nanometers, to be obtained from a composition based on a polymeric compound to be electrospun that is subjected to an electric field is known. Depending on the type of compound being electrospun, the nanofibers obtained can then have applications in any industry, for example in medicine, military defense, environment, biotechnology, energy or in the cosmetic industry.

For environmental reasons, electrospinning tends to be performed using environmentally friendly solvents, especially in water. However, electrospinning of polymers in pure water is not simple because of its surface tension, and the viscosity of the compound that is obtained.

To overcome this problem, an attempt was made to lower the surface tension and viscosity of water, for example by electrospinning in aqueous ammonium solutions, then in high pH solutions, or even in water in the presence of dimethylformamide DMF at 40° C. Tests were also performed with hexafluoroisopropanol HFIP or ethanol. In doing so, however, the ecological aspect is lost.

Compositions to be electrospun based on a biocompatible polymer also comprising an electrospinning promoter have been proposed which make electrospinning in water possible. But with the compositions described so far, the production of fibers on an industrial scale can be further improved, especially in terms of productivity.

There is therefore the need to perfect a composition to be electrospun that can overcome at least one of the disadvantages of the state of the art.

To do this, it is necessary to solve the technical problem of a composition to be electrospun that allows the production, by means of electrospinning and on an industrial scale, of an electrospun fiber that does not have an environmental impact.

In particular, one purpose of the present invention is to provide a composition to be electrospun which can be electrospun in water and on an industrial scale with acceptable yields.

Another purpose of the present invention is to provide a composition to be electrospun which allows continuous nanofibers, or in any case fibers, to be produced with constant or almost constant diameter and without defects.

Another purpose is to develop a method for preparing the above-mentioned composition to be electrospun.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes, and to resolve the technical problem disclosed above in a new and original way, also achieving considerable advantages compared to the state of the prior art, a composition to be electrospun according to the present invention comprises a first compound to be electrospun, a spinning promoter and a surfactant. The function of the spinning promoter is to facilitate spinning of the first compound, in particular to stabilize the electrospinning process so as to obtain regular fibers. The surfactant instead has the function of improving the electrospinning process in order to allow higher delivery rates and thus have higher yields, while avoiding the formation of defects in the fibers. This improvement is achieved through the reduction of the surface tension of the composition which has also reduced its conductivity. In particular, it has been observed that the surfactant makes it possible to avoid the phenomenon of dripping during electrospinning.

The first compound to be electrospun is a biocompatible polymer suitable to be electrospun and selected from a group consisting of a first polysaccharide, collagen, gelatin, albumin, elastin and their derivatives. Advantageously, the first polysaccharide is selected from a group consisting of xanthan gum, pectins, chitin, chitosan, dextran, carrageenan, guar gum, agar, cellulose derivatives, starch, gelatin, μ-glucans, glycosaminoglycans, mucopolysaccharides, water-soluble polysaccharides and their derivatives.

Preferably, the cellulose derivatives are selected from hydroxypropylmethyl cellulose HPMC, hydroxypropyl cellulose HPC, hydroxyethyl cellulose HEC, sodium carboxymethyl cellulose Na-CMC. The glycosaminoglycans GAG or mucopolysaccharides may be chosen from chondroitin sulfate, dermatan sulfate, heparin, heparan sulfate and hyaluronic acid HA. Water-soluble polysaccharides can be chosen from galactomannans, xylans, gum arabic, gum ghatti, glucomannan, acemannan, soluble dietary fibers, glycogen, amyloses and polysaccharides derived from plants, bacteria and fungi.

The spinning promoter is a polymer carrier, possibly also uncharged, selected from a group consisting of a second polysaccharide, chemically different from the first polysaccharide, possibly in the presence of poly(oxyethylene) PEO. Advantageously, the polymer carrier, possibly even without charge, is also biocompatible.

Preferably, the electrospinning promoter is chosen from pullulan and alginate, possibly mixed with poly(oxyethylene). The promoter may also comprise a mixture of pullulan and alginate. More preferably, the promoter is chemically different from the first compound to be electrospun, i.e., the first compound to be electrospun is not pullulan or alginate.

The surfactant is advantageously a biosurfactant, i.e., a surfactant of biological and organic origin. In particular, the surfactant is obtained by fermentation of gram positive microorganisms. Such a surfactant has biological origins, which makes it biocompatible.

Preferably, the surfactant is chosen among the lipopeptides, in particular obtained from Bacillus sp. More preferably the surfactant is chosen from the surfactin family, in particular from the group consisting of surfactins, pumilacidins and lichenysins.

Advantageously the surfactant is concentrated between 0.1 and 5%, more advantageously between 0.2 and 2%, even more advantageously between 0.5 and 1% by weight of the total weight of the composition.

In accordance with embodiments the pH of the composition is maintained at slightly basic values, in particular between 6 and 7. To do this it is appropriate to add a basic compound to the composition, for example NaOH.

According to embodiments, the composition also comprises an active ingredient. Such an active ingredient may, for example, be chosen from cosmetic active ingredients, pharmaceutical active ingredients and nutritional active ingredients.

According to embodiments, the composition also comprises a stabilizer, suitable to improve the stability of the fibers obtained after electrospinning. Preferably the stabilizer is a cross-linkable polymer.

An advantage of the above composition lies in the possibility of making a cosmetic, pharmaceutical or nutritional product based on natural polymers, with topical concentrations of the compound of interest much higher than those obtainable with traditional formulations. This is due to the fact that the compound of interest is precisely integrated into the structure of the final product. With the known compositions, it is practically impossible to reach high concentrations, even with polysaccharides, collagen, gelatin, albumin, elastin and their low molecular weight derivatives, since the viscosity of the product increases too much and it is not possible to exceed 5-10% by weight. With the present composition, products can be obtained which allow the application of concentrations up to 50% by weight of the compound of interest.

Note that with the composition according to the present invention, it is possible to envisage different types of use depending on the electrospun compound but also depending on the active ingredient; in particular it is possible to provide uses in the cosmetic, pharmaceutical or nutritional industries. More precisely, cosmetic use may provide for topical skin applications as mentioned above, pharmaceutical and nutritional uses may instead provide for the release of active ingredients, drugs and/or supplements in the body.

According to one aspect, there is also provided a method for preparing a composition to be electrospun, wherein a first compound to be electrospun, a spinning promoter, and a surfactant are mixed.

The first compound to be electrospun is a biocompatible polymer suitable to be electrospun and is selected from a group consisting of a first polysaccharide, collagen, albumin, gelatin, elastin and derivatives thereof. Advantageously, the first polysaccharide is chosen from a group consisting of xanthan gum, pectins, chitin, chitosan, dextran, carrageenan, guar gum, agar, cellulose derivatives, starch, μ-glucans, glycosaminoglycans, mucopolysaccharides, water-soluble polysaccharides and their derivatives. Preferably, the cellulose derivatives are selected from hydroxypropylmethylcellulose HPMC, hydroxypropylcellulose HPC, hydroxyethylcellulose HEC, sodium carboxymethylcellulose Na-CMC. The glycosaminoglycans GAG or mucopolysaccharides may be chosen from chondroitin sulfate, dermatan sulfate, heparin, heparan sulfate and hyaluronic acid HA. Water-soluble polysaccharides can be chosen from galactomannans, xylans, gum arabic, gum ghatti, glucomannan, acemannan, soluble dietary fibers, glycogen, amylose and polysaccharides derived from plants, bacteria and fungi.

The spinning promoter is an uncharged polymer carrier selected from a group consisting of a second polysaccharide, chemically different from the first polysaccharide, possibly in the presence of poly(oxyethylene) PEO. Favorably, the uncharged polymer carrier is also biocompatible. Preferably the second polysaccharide of the composition is selected from alginate and pullulan.

The surfactant is a biosurfactant, i.e., a surfactant of biological and organic origin. In particular, the biosurfactant is obtained by fermentation of gram positive microorganisms. Such a surfactant has biological origins, which makes it biocompatible.

Preferably, the surfactant is chosen among the lipopeptides, in particular obtained from Bacillus sp. More preferably, the surfactant is chosen from the surfactins family, particularly in the group consisting of surfactins, pumilacidins and lichenysins.

In accordance with embodiments, the method involves adding the surfactant before starting the electrospinning of the composition, so as to stabilize the production. For example, the method may involve dissolving the first compound, the electrospinning promoter and the surfactant in water, preferably in respective separate aliquots of water, and mixing them.

In accordance with embodiments the method envisages keeping the pH of the composition at a slightly basic value, i.e., between 6 and 7, by addition of a basic compound. The latter may be any base, for example, NaOH.

According to embodiments, the method comprises adding an active ingredient. Such an active ingredient may, for example, be chosen from cosmetic active ingredients, pharmaceutical active ingredients and nutritional active ingredients.

According to embodiments, the method also provides for adding a stabilizer, which is suitable to improve the stability of the fibers obtained after electrospinning. Preferably the stabilizer is a cross-linkable polymer.

According to another aspect, a patch product is provided, to be applied preferentially to the skin, composed of a membrane substrate absorbable by the skin. The membrane substrate is electrospun. The electrospun membrane substrate is composed of at least one fiber, preferably a nanofiber, in particular in the form of a non-woven fabric.

The at least one fiber forming the membrane substrate is, in turn, comprised of a first electrospun compound, an electrospinning promoter, and a surfactant. These compounds are of the type defined above for the composition and for the method.

In accordance with embodiments, the fiber has a flat-shaped, i.e., flattened, section.

### DESCRIPTION OF SOME EMBODIMENTS OF THE PRESENT INVENTION

Unless defined otherwise, all technical and scientific terms used herein and below have the same meaning as commonly understood by one of ordinary skill in the art to which the present invention belongs. Although methods and materials similar or equivalent to those described herein can be used in the practice or testing of the present disclosure, the methods and materials are described, by way of example, below. In case of conflict, this application, including the definitions, takes precedence. Materials, methods and examples are intended to be illustrative only and are not to be construed as limiting.

All measurements are carried out at 25° C. (room temperature) and at atmospheric pressure, unless otherwise indicated. All temperatures are in degrees Celsius unless otherwise indicated.

All percentages and ratios indicated are intended to refer to the weight of the total composition (w/w), unless otherwise indicated.

All the percentage ranges shown here are provided with the expectation that the sum with respect to the total composition is 100%, unless otherwise indicated.

All ranges set forth herein are intended to include extremes, including those that report a range “between” two values, unless otherwise indicated.

Also included in the present disclosure are ranges that result from overlapping or joining two or more disclosed ranges, unless otherwise indicated.

Also included in the present disclosure are ranges that may result from the combination of two or more disclosed point values, unless otherwise indicated.

Where water is mentioned, it is distilled water unless otherwise specified.

The composition comprises a first compound to be electrospun, which is a biocompatible polymer suitable to be electrospun and is chosen from xanthan gum, pectins, chitin, chitosan, dextran, carrageenan, guar gum, agar, hydroxypropylmethylcellulose HPMC, hydroxypropylcellulose HPC, hydroxyethylcellulose HEC, sodium carboxymethylcellulose Na-CMC, albumin, starch, gelatin, collagen, elastin, μ-glucans, chondroitin sulfate, dermatan sulfate, heparin, heparan sulfate, hyaluronic acid HA, galactomannans, xylans, gum arabic, gum ghatti, glucomannan, acemannan, soluble dietary fibers, glycogen, amylose and polysaccharides derived from plants, bacteria and fungi and derivatives thereof.

Such compounds or classes of compounds have as their properties the possibility of modifying the viscosity of liquids, which makes them suitable for forming regular electrospun fibers with good mechanical and absorption properties. They are also all biocompatible, of natural origin, and can be used in the food, pharmaceutical and/or cosmetic industry.

In particular, xanthan gum, dextran, carrageenan, Na-CMC, starch, gelatin and gum ghatti are used as a thickener, stabilizer and possibly also as a gelling agent in the food industry. In addition, xanthan gum is used as a stabilizer for suspensions and emulsions in the pharmaceutical and cosmetic industry, guar gum is used as a thickener and gelling agent also in the pharmaceutical and cosmetic industry, carrageenan is used as an inactive excipient in the pharmaceutical industry. Dextran is also used as a thickener in the pharmaceutical industry.

Chitosan is used in the food industry, in low-calorie diets, and in the pharmaceutical industry as an excipient, particularly for inhaled products. Pectin, on the other hand, is used as a gelling agent in the food industry and as a dietary agent and probiotic in the pharmaceutical industry.

Agar agar, galactomannans and glucomannan are used as gelling agents in the nutritional industry.

Among cellulose derivatives, HPMC is used as a stabilizer and viscosity regulator in the food industry, and as eye drops or an excipient for oral medicinal products in pharmaceuticals. HPC is used as a food additive, and as eye drops or as a binder for tablets in the pharmaceutical industry. HEC is used as a thickener and gelling agent in the pharmaceutical and cosmetic industries.

μ-glucans are used as dietary fibers, as are xylans. Chondroitin sulfate is used as a dietary supplement, and also in the treatment of osteoarthritis symptoms. Dermatan sulphate, heparin, and heparan sulfate are known as anticoagulants in the pharmaceutical industry.

Gum arabic is used in the food industry as a stabilizing excipient and viscosity modifier. Acemannan is known in the pharmaceutical industry for its immunostimulatory properties.

It should be noted that some of these compounds have their own functions in the cosmetic, pharmaceutical or food industry, such as starch, elastin, hyaluronic acid, heparin, collagen, pectin, μ-glucans, chondroitin sulfate, dermatan sulfate, heparan sulfate and their derivatives, among others. It is therefore advantageous to electrospin these compounds, since the application of the corresponding fibers will allow these compounds to be applied in higher doses than the known solutions, for the benefit of their greater effectiveness.

Where the first compound to be electrospun is hyaluronic acid, it may be linear or cross-linked, and may have a high mass, for example of the order of one million Daltons or even more, or alternatively have a low mass, typically of the order of 10000 Daltons or less. It is also possible to provide a mixture of linear hyaluronic acid with cross-linked hyaluronic acid, so as to modulate the rigidity of the yarn to be obtained as well as the three-dimensional structure of a film obtainable by depositing the yarn on a support.

The first compound is advantageously diluted in an aqueous or aqueous-based solution, at concentrations of for example between 0.1% and 30% by weight, preferably between 1% and 20% by weight, more preferably between 5% and 15% by weight.

The composition to be electrospun also includes a spinning promoter which is a favorably biocompatible, uncharged polymer carrier. It is chosen from alginate, possibly in the presence of PEO, or pullulan. The spinning promoter is preferably pullulan, as it allows to obtain the best results.

In case the alginate is mixed with the PEO, they can be diluted separately in aqueous or aqueous-based solutions, at a concentration of between 0.1% and 30% by weight, preferably between 1% and 20% by weight, more preferably between 5% and 15% by weight. The alginate:PEO mixture, if the latter is present, is made in weight proportions preferably ranging from 5:1 to 1:5, more preferably from 2:1 to 1:2. The best electrospinning results were obtained with proportions equal to 1:1 by weight.

The pullulan may be diluted in an aqueous-based or aqueous solution preferably in a concentration of from 0.1% to 30% by weight, preferably from 1% to 20% by weight, more preferably from 5% to 15% by weight.

The first compound to be electrospun and the promoter are mixed in proportions (first compound): promoter preferably from 10:1 to 1:10, more preferably from 5:1 to 1:5, even more preferably from 2:1 to 1:2 by weight. It has been found that the best results are obtainable with compositions in which the weight ratio of first compound to promoter is 1:1.

The composition also comprises a surfactant, chosen from the biosurfactants, i.e., surfactants having biological and organic origin, as opposed to surfactants of bacterial origin.

In particular, the surfactants of the composition are advantageously obtained by fermentation of gram positive microorganisms.

More precisely the biosurfactant is chosen from lipopeptides, in particular those derived from the fermentation of organisms of the species Bacillus, more particularly those of low molecular weight, for example whose molecular weight is at most 10000 g/mol, preferably at most 5000 g/mol, more preferably at most 2500 g/mol.

Among these lipopeptides are several families, such as surfactins, iturins, fengycins, kurstatins and locillomycins. These families of lipopeptides are distinguished from one another by the type and sequence of amino acid residues, the nature of the peptide cycle, and the nature, length, and branching of the fatty acid chain. All, however, have properties of surfactants.

Preferably, the biosurfactant of the composition is selected from the family of surfactins.

In the surfactins family there are the surfactins themselves, which can be produced by different branches of the Bacillus species, especially B. subtilis, but also others such as B. natto or B. circulans. Their structure comprises a lactone ring formed of seven amino acids with a characteristic chiral sequence (L-Glu, L-Leu, D-Leu, L-Val, L-Asp, D-Leu and L-Leu wherein the first and last amino acids are linked by an ester linkage to form the lactone ring). Surfactins have a μ-hydroxy fatty acid chain with lengths from thirteen to sixteen carbon atoms. Although more than 30 congeners are known among the surfactins, they all have the same chiral LLDLLDL sequence of amino acids in the lactone ring. Congeners are molecules of the same genus but not isomers.

The family of surfactins also includes pumilacidins, obtainable from the B. pumilus branch with variants A-F. Compared to surfactins, the amino acids in position 7 are substituted with L-Val or L-Ile.

The family of surfactins finally includes lichenysins, obtained mainly from B. licheniformis, which differ from the surfactins by the substitution in position 1 of the lactone ring of L-glutamic acid with L-glutamine.

Surfactins, pumilacidins and lichenysins include five hydrophobic residues (Leu, Val or Ile) and a long secondary aliphatic chain, which give them powerful surfactant properties. In fact, it has been verified that a compound of the surfactins family at high purity is able to lower the surface tension of water from 72 to 27 mN/m at a concentration of 0.0005% by weight. Surfactins are stable and soluble in water over a wide range of pH (5-13) and temperatures, making them particularly suitable to be electrospun in aqueous solutions.

Preferably the biosurfactant is a surfactin. In particular, the surfactin, or biosurfactin, may have a solubility of 548 g/L in water at 20° C., a critical micelle concentration (CMC) of 0.0003% by weight, a ratio of hydrophilic groups to hydrophobic groups (HLB) of from 10 to 17, more preferably from 15.9 to 16.5 (considering a chain of μ-hydroxy fatty acids with length C₁₃-C₁₅) or from 10 to 12, a critical packaging parameter (CPP) of less than 0.33, more preferably from 0.12 to 0.16, and/or pKa values of from 4.271 to 3.727.

It should be noted that the above HLB value has been calculated using the Griffin method.

Generally, the biosurfactant is present at a concentration of less than 5%, preferably less than 2%, more preferably between 0.5% and 1% by weight based on the weight of the composition.

The composition also includes at least one active ingredient, of the pharmaceutical, nutritional and/or cosmetic type.

It should be noted that the active ingredients can have various types of function, regardless of their field of action.

The cosmetic active ingredient may be of the following types: anti-seborrhoeic (for example sebacic acid, azelaic acid), anti-sebum (for example charcoal powder), antimicrobial (for example climbazole, pyroctone olamine), antioxidant (for example ascorbic acid, tocopherol, co-enzyme Q10, resveratrol, glutathione), antiperspirant (for example aluminium chlorohydrate, aluminium sesquichlorohydrate), astringent (for example Citrus aurantifolia flower extract, calcium lactate), bleach (for example glabridine, ammonium persulfate), cleanser (for example sodium cocoyl glutamate), deodorant (for example triethyl citrate, zinc ricinoleate), exfoliant (for example glycolic acid, malic acid, mandelic acid), flavourings (for example for example citral, honey), fragrance (for example d, 1-limonene, coumarin), humectant (for example glycerin, propanediol), keratolytic (for example chloroacetic acid, salicylic acid), moisturiser (for example Aloe arborescens leaf extract), perfumant (for example geraniol, linalool), emollient (for example triolein, squalene), refreshant (for example menthol, menthyl lactate), skin moisturiser (for example panthenol, allantoin), skin protection (for example sphingolipids, zinc oxide), smoothing agent (for example Ricinus communis seed oil), soothing agent (for example Hamamelis virginiana extracts, Camomilla extracts rictica, bisabolol) or tonic (for example arnica montana, Capsicum frutescens extract), UV filter (for example methylene bis-benzotriazolyl tetramethylbutylphenol, ethylhexyl methoxycinnamate, caffeine, theine, theobromine, theophylline).

The pharmaceutical active ingredient can be of the following types: 5-alpha-reductase inhibitor (e.g., finasteride), 5-aminosalicylates (e.g., mesalamine), 5HT3 receptor antagonist (e.g., ondansetrone), ACE inhibitor with calcium antagonist agent (e.g., amlodipine/benazepril), ACE inhibitor with thiazide (e.g., hydrochlorothiazide), adamantane antivirals (e.g., amantadine), adrenal corticosteroid inhibitor (e.g., aminoglutethimide), adrenergic bronchodilators (e.g., albuterol), agent for hypertensive emergencies (e.g., diazoxide), agent for pulmonary hypertension (e.g., treprostinil), aldosterone receptor antagonist (e.g., spironolactone), alkylating agent (e.g., cyclophosphamide), allergens (e.g., house dust mite allergen extracts), alpha-glucosidase inhibitor (e.g., miglitol), amebicides (e.g., metronidazole), aminoglycosides (e.g., tobramiycin), aminopenicillins (e.g., amoxicillin), aminosalicylates (e.g., aminosalicylic acid), AMPA receptor antagonist (e.g., perampanel), amylin analogues (e.g., pramlintida), analgesics (e.g., acetaminophen), androgenic and anabolic steroids (e.g., testosterone) angiotensin-converting enzyme inhibitor (e.g., ramipril), angiotensin II inhibitor with calcium channel blocker (e.g., amlodipine/olmesartan), angiotensin II inhibitor with thiazides (e.g., hydrochlorothiazide/olmesartan), angiotensin receptor blockers (e.g., valsartan), angiotensin and neprilysin receptor blocker inhibitor (e.g., sacubitril/valsartan), anorectal preparations (e.g., hydrocortisone/pramoxin), anorectics (e.g., phentermine), antacids (e.g., magnesium hydroxide), anthelmintics (e.g., pyrantel), anti-angiogenic ophthalmic agents (e.g., aflibercept), anti-CTLA-4 monoclonal antibody (e.g., ipilimumab), monoclonal anti-PD-1 antibody (e.g., nivolumab), anti-adrenergic (central) agent with thiazides (e.g., hydrochlorothiazide/methyldopa), anti-adrenergic (peripheral) agent with thiazides (e.g., polythiazide/prazosine) centrally acting anti-adrenergic agent (e.g., guanfacine), peripherally acting anti-adrenergic agent (e.g., tamsulosin), antiandrogens (e.g., enzalutamide), anti-anginal agent (e.g., nitroglycerin), e.g., diphylline/guaifenesin), antibiotics (e.g., metronidazole), antibiotics/antineoplastics (e.g., doxorubicin), anticholinergic antiemetics (e.g., diphenhydramine), anticholinergic antiparkinsonian agent (e.g., procyclidine), anticholinergic bronchodilators (e.g., tiotropicin), e.g., anticholinergics/antispasmodics (e.g., hyoscyamine), anticoagulant agent (e.g., phytonadione), anticonvulsant (e.g., lacosamide), antidepressant (e.g., bupropion), antidiarrhoeic (e.g., loperamide), antidiuretic hormone (e.g., desmopressin), antidote (e.g., naltrexone dronabinol), antifungal (e.g., griseofulvin), antigonadotropic agent (e.g., danazol), antigout agent (e.g., colchicine), antihistamine (e.g., cetirizine), anti-hyperlipidaemic agent and combinations (e.g., ezetimibe/simvastatin), anti-hypercaemic agent (e.g., febuxostat), antimalarial agent (e.g., doxycycline), antimalarial combination, antimalarial quinoline (e.g., hydroxychloroquine), antimanic agent (e.g., lithium), antimetabolite (e.g., capecitabine), antiemycranic agent (e.g., rizatriptan), antineoplastic agent (e.g., isotretinoin), antineoplastic combination (e.g., letrozole/ribociclib), antineoplastic detoxifying agent (e.g., amifostine), antineoplastic interferon (e.g., interferon alpha-2b), antipseudomonal penicillin (e.g., carbenicillin), antipsoriatic (e.g., acitretin), antipsychotic agent (e.g., haloperidol), anti-rheumatic (e.g., adalimumab), antiseptic and germicide, antithyroid agent (e.g., potassium iodide), antitoxin and antiviral (e.g., polyvalent antivenin (crotalidae)), antitussive (e.g., dextromethorphan), antiviral booster (e.g., ritonavir), antiviral interferon (e.g., peginterferon alpha-2a), aromatase inhibitor (e.g., anastrozole), atypical antipsychotic (e.g., aripiprazole), azole antimycotic (e.g., fluconazole), bacterial vaccine (e.g., 13-valent pneumococcal vaccine), anticonvulsant barbiturate (e.g., primidone), barbiturate (e.g., phenobarbital), BCR-ABL tyrosine kinase inhibitor (e.g., imatin), anticonvulsant benzodiazepine (e.g., diazepam), benzodiazepine (e.g., clonazepam), beta-blocker with thiazides (e.g., bisoprolol/hydrochlorothiazide), beta-lactamase inhibitor (e.g., clavulanic acid), bile acid sequestrant (e.g., colesevelam), bisphosphonate (e.g., zoledronic acid), BTK inhibitor (e.g., ibrutinib), calcimimetic (e.g., cinacalcet), calcineurin inhibitor (e.g., tacrolimus), calcitonin, calcium channel blocking agent (e.g., verapamil), carbamate anticonvulsant (e.g., felbamate), carbapenem (e.g., doripenem), carbapenem/beta-lactamase inhibitor (e.g., meropenem/vaborbactam) anticonvulsant carbonic anhydrase inhibitor (e.g., topiramate), carbonic anhydrase inhibitor (e.g., acetazolamide), regenosadase stress agent, beta-cardioselective beta-blocker (e.g., nebivolol), catecholamines (e.g., epinephrine), CD20 monoclonal antibody (e.g., ocrelizumab), CD30 monoclonal antibody (e.g., brentuximab), CD33 monoclonal antibody (e.g., gemtuzumab), CD38 monoclonal antibody (e.g., CD52 monoclonal), (e.g., alemtuzumab), CDK 4/6 inhibitor (e.g., palbociclib), cephalosporins/beta-lactamase inhibitor (e.g., avibactam/ceftazidime), cerumenolytics (e.g., carbamide peroxide), CFTR combination (e.g., ivacaftor/lumacaftor), CFTR enhancer (e.g., ivacaftor), CGRP inhibitor (e.g., erenumab), chelating agent (e.g., deferasirox), chemokine receptor antagonist (e.g., maraviroc), chloride channel activator (e.g., lubiprostone), cholesterol absorption inhibitor (e.g., ezetimibe), cholinergic agonist (e.g., cevimeline), cholinergic muscle stimulant (e.g., pyridostigmine), cholinesterase inhibitor (e.g., donepezil), central nervous system stimulant (e.g., phentermine), colony-stimulating factor (e.g., filgrastim), contraceptive (e.g., levonorgestrel), corticotropin, coumarins and indandions (e.g., warfarin), cox-2 inhibitor (e.g., celecoxib), decongestant (e.g., pseudoephedrine), diarylquinoline (e.g.,), anticonvulsant dibenzazepine (e.g., carbamazepine), digestive enzyme (e.g., lactase), dipeptidyl peptidase 4 inhibitor (e.g., sitagliptin), dopaminergic antiparkinsonian agent (e.g., ropinirole), drug used in alcohol dependence (e.g., acamprosate), echinocandins (e.g., caspofungin) inhibitor (e.g., erlotinib), oestrogen receptor antagonist (e.g., fulvestrant), oestrogen (e.g., oestradiol), expectorant (e.g., guaifenesin), factor Xa inhibitor (e.g., rivaroxaban), anticonvulsant fatty acid derivative (e.g., divalproex sodium), fibric acid derivative (e.g., fenofibrate), first-generation cephalosporins (e.g., cephalexin), fourth-generation cephalosporins (e.g., cefepime), gallstone-solubilising agent (e.g., ursodiol), gamma-aminobutyric acid analogue (e.g., gabapentin), gamma-aminobutyric acid reuptake inhibitor (e.g., tiagabine), general anaesthetic (e.g., propofol), GI stimulant (e.g., metoclopramide), glucocorticoids (e.g., budesonide), glucose elevating agent (e.g., glucagon), antibiotic (e.g., vancomycin), platelet glycoprotein inhibitor (e.g., tirofiban), glycylcycline (e.g., tigecycline), gonadotropin-releasing hormone (e.g., leuprolide), gonadotropin-releasing hormone antagonist (e.g., elagolix), gonadotropin (e.g., chorionic gonadotropin) group I antiarrhythmic (e.g., phenytoin), group II antiarrhythmic (e.g., propranolol), group III antiarrhythmic (e.g., dronedarone), group IV antiarrhythmic (e.g., verapamil), group V antiarrhythmic (e.g., digoxin), growth hormone receptor blocker (e.g., pegvisomant), growth hormone (e.g., somatropin), guanylate cyclase-C agonist (e.g., linaclotide), H. pylori eradication agent (e.g., bismuth subcitrate potassium/metronidazole/tetracyclines), H2 antagonist (e.g., ranitidine), hedgehog pathway inhibitor (e.g., vismodegib), heparin antagonist (e.g., protamine), HER2 inhibitor (e.g., neratinib) herbal product (e.g., 5-hydroxytryptophan, aloe vera), histone deacetylase inhibitor (e.g., romidepsin), hormone/antineoplastic (e.g., medroxyprogesterone), hydantoin anticonvulsant (e.g., phenytoin), hydrazide derivative (e.g., isoniazid), immunoglobulin, impotence agent (e.g., sildenafil), incretin mimetic (e.g., liraglutide), inotropic agent (e.g., digoxin), insulin and its derivatives (e.g., insulin glargine), insulin-like growth factor (e.g., mecasermine), interferon (e.g., interferon beta-la), interleukin inhibitor (e.g., dupilumab), interleukin (e.g., aldesleukin), iron product (e.g., ferrous sulphate), ketolide (e.g., telithromycin) laxative (e.g., bisacodyl), leprostatic (e.g., clofazimine), leukotriene modifier (e.g., montelukast), lincomycin derivative (e.g., clindamycin), loop diuretic (e.g., furosemide), lysosomal enzyme (e.g., imiglucerase), macrolide (e.g., azithromycin), mast cell stabilizer (e.g., cromolyn), meglitinide (e.g., repaglinide), melanocortin receptor agonist (e.g., bremelanotide), methylxanthine (e.g., theocortic) mineral corticoid (e.g., fludrocortisone), mineral and electrolyte (e.g., citric acid/potassium citrate), various antivirals (e.g., baloxavir marboxil), various anxiolytics, sedatives and hypnotics (e.g., zolpidem), various bone resorption inhibitors (e.g., denosumab), various cardiovascular agents (e.g., midodrine), various agents of the central nervous system (e.g., dalfampridine), various coagulation modifiers (e.g., tranexamic acid), various diuretics (e.g., pamabrom), various agents of the genito-urinary tract (e.g., phenazopyridine), various gastrointestinal agents (e.g., misoprostol), various metabolic agents (e.g., burosumab), various respiratory agents (e.g., alpha 1-proteinase inhibitor), various topical agents (e.g., sodium hyaluronate), various vaginal agents (e.g., oestradiol), mitotic inhibitor (e.g., vincristine), monoamine oxidase inhibitor (e.g., phenelzine), mouth and throat product (e.g., fluoride), mTOR inhibitor (e.g., everolimus), mucolytic (e.g., acetylcysteine), multichinase inhibitor (e.g., sorafenib), narcotic analgesic combination (e.g., buprenorphine/naloxone), narcotic analgesic (e.g., fentanyl), natural penicillin (e.g., penicillin v potassium), neuraminidase inhibitor (e.g., oseltamivir), neuronal potassium channel opener (e.g., ezogabine), new generation cephalosporin (e.g., ceftaroline), NHE3 inhibitor (e.g., ceftaroline), nicotinic acid derivative (e.g., ethionamide), NK1 receptor antagonist (e.g., aprepitant), NNRTI (e.g., efavirenz), non-cardioselective beta-blocker (e.g., carvedilol), non-sulphonylureas (e.g., metformin), non-steroidal anti-inflammatory drug (e.g., diclofenac), NS5A inhibitor (e.g., daclatasvir), nucleoside reverse transcriptase inhibitor (NRTI) (e.g., tenofovir), nutraceutical (e.g., omega-3 polyunsaturated fatty acids), oral nutritional supplement (e.g., arginine), other immunostimulants (e.g., glatiramer), other immunosuppressants (e.g., omalizumab), anticonvulsant oxazolidinedione (e.g., trimethadione), antibiotic oxazolidinone (e.g., linezolid), parathormone and analogues (e.g., teriparatide), PARP inhibitor (e.g., niraparib), PCSK9 inhibitor (e.g., evolocumab), penicillinase-resistant penicillin (e.g., oxacillin), peripheral opioid receptor antagonist (e.g., naloxegol), mixed peripheral opioid receptor agonists (eluxadoline hegonist/antagonist), peripheral vasodilator (e.g., isoxsuprine), peripherally acting anti-obesity agent (e.g., orlistat), phenothiazine antiemetic (e.g., promethazine), phenothiazine antipsychotic (e.g., prochlorperazine), phenylpiperazine antidepressant (e.g., trazodone), potassium phosphate inhibitor (e.g., trazoderrone) (e.g., idelalisib), platelet aggregation inhibitor (e.g., aspirin), platelet-stimulating agent (e.g., eltrombopag), polyene (e.g., nystatin), potassium-sparing diuretic (e.g., spironolactone), probiotic (e.g., Lactobacillus acidophilus), progesterone receptor modulator (e.g., ulipristal), progestin levonorgestrel), prolactin inhibitor (e.g., cabergoline), protease inhibitor (e.g., telaprevir), protease-activated receptor-1 antagonist (e.g., vorapaxar), proteasome inhibitor (e.g., bortezomib), proton pump inhibitor (e.g., omeprazole), psoralen (e.g., methoxsalen), purine nucleoside (e.g., valacyclovir), pyrrolidine anticonvulsant (e.g., levetiracetam), quinolones (e.g., ciprofloxacin), recombinant human erythropoietin (e.g., epoetin alpha), renin inhibitor (e.g., aliskiren), rifamycin derivative (e.g., rifampicin), salicylate (e.g., aspirin), second-generation cephalosporin (e.g., cefuroxime receptor selective), modulator (e.g., ospemifene), selective immunosuppressant (e.g., natalizumab), selective phosphodiesterase-4 inhibitor (e.g., roflumilast), selective serotonin reuptake inhibitor (e.g., escitalopram), serotonin-norepinephrine reuptake inhibitor (e.g., duloxetine), serotoninergic neuroenteric modulator (e.g., tegaserod), SGLT-2 inhibitor (e.g., empagliflozin), skeletal muscle relaxant (e.g., onabotulinumtoxinA), smoking cessation agent (e.g., nicotine analogue somatostat) (e.g., octreotide), statin (e.g., lovastatin), streptogramin (e.g., dalfopristin/quinupristin), streptomycete derivative (e.g., capreomycin), anticonvulsant succinimide (e.g., ethosuximide), sulphonamide (e.g., sulfamethoxazole), stimulant sulphonylurea (e.g., glimepistole) clomiphene), tetracyclic antidepressant (e.g., mirtazapine), tetracyclines (e.g., minocycline), thiazide diuretic (e.g., hydrochlorothiazide), thiazolidinedione (e.g., pioglitazone), thioxanthene (e.g., thiotixene), third-generation cephalosporin (e.g., ceftriaxone), thrombin inhibitor (e.g., dabigatazone), strepolytic (e.g., levothyroxine), TNF alpha inhibitor (e.g., adalimumab), tocolytic agent (e.g., terbutaline), topical agent for acne (e.g., tretinoin), topical anaesthetic (e.g., lidocaine), topical anti-infective (e.g., malathion), topical anti-rosaceous agent (e.g., ivermectin), topical antibiotic (e.g., silver sulfadiazine), topical antifungal (e.g., econazole), topical antihistamine (e.g., diphenhydramine), topical antineoplastic (e.g., imiquimod), topical antipsoriatic (e.g., tazarotene) topical antiviral (e.g., penciclovir), topical astringent (e.g., hazelnut), topical debriding agent (e.g., collagenase), topical depigmenting agent (e.g., hydroquinone), topical emollient (e.g., emollients), topical keratolytics (e.g., salicylic acid), topical non-steroidal anti-inflammatory agent (e.g., diclofenac), topical photochemical (e.g., aminolevulinic acid), topical rubefacient (e.g., menthol), topical steroid (e.g., betamethasone), topical steroid with anti-infectives (e.g., acyclovir/hydrocortisone), transthyretin stabiliser (e.g., tafamidis), triazine anticonvulsant (e.g., lamotrigine), tricyclic antidepressant (e.g., amitriptyline), urea cycle disrupting agent (e.g., sodium phenylbutyrate), urinary anti-infective (e.g., nitrofurantoin), urinary antispasmodic (e.g., amitriptyline) modifier (e.g., potassium citrate), uterotonic agent (e.g., dinoprostone), vaginal anti-infective (e.g., clindamycin), vasodilator (e.g., alprostadil) vasopressin antagonist (e.g., conivaptan), vasopressor (e.g., epinephrine), VEGF/VEGFR inhibitor (e.g., pazopan), viral vaccine, vitamin/mineral combination, vitamin (e.g., cyanocobalamin), VMAT2 inhibitor (e.g., valbenazine).

The nutritional active ingredient can be of the vitamin type (e.g., vitamins A, B, C, D, E, K, folic acid, biotin), mineral (e.g., potassium, chlorine, sodium, calcium, phosphorus, magnesium, iron, zinc, manganese, copper, iodine, chromium, molybdenum, selenium, cobalt, fluoride), amino acid, peptide and protein and their metabolites and derivatives (for example essential and branched amino acids, carnosine, enzymes and enzyme complexes, lactoferrin, N-acetylcysteine, animal or vegetable food proteins), fatty acid (for example omega-3, omega-6, omega-9 fatty acids), natural product manufactured using intact sources or extracted or derived from plants, animals, algae, fungi, lichens or bacteria (for example phytosterol, echinacea, green tea extract, garlic, aloe vera, fish oil, spirulina, chlorella, digestive enzymes derived from fungi), sugar and polysaccharide (e.g., mannose, ribose, trehalose, dextrose, glucuronolactone, dextrins), probiotic (e.g., live microorganisms such as Lactobacillus spp, Bifidobacterium spp, Sacc boulardii), prebiotic (e.g., fructans such as fructooligosaccharides and inulins, galactans such as galactooligosaccharides and xylooligosaccharides), antioxidant (e.g., lipoic acid, coenzyme Q10, flavono, glutathione, resveratrol, catechins), other substances with a nutritional or physiological effect, e.g., betaine, caffeine theobromine, theophylline, CDP-choline, choline, creatine, phospholipids, GABA, glucosamine, inositol, melatonin, methylsulphonylmethane, nucleotides, squalene).

Inclusion of the active ingredient in the electrospun fiber may be achieved by co-electrospinning the active ingredient with the first compound. In this case, a mixture of the first compound to be electrospun and the electrospinning promoter with the active ingredient can be prepared, and the obtained mixture is electrospun.

Alternatively, it may be envisaged to electrospin the first compound alone, and subsequently to integrate the active ingredient into the obtained fiber. Depending on the applications, it can be expected that the active ingredient is absorbed in the electrospun fiber, or that it is trapped in the three-dimensional structure obtained with the electrospun fiber.

For example, the first compound to be electrospun may be composed of a mixture of linear hyaluronic acid with cross-linked hyaluronic acid. The cross-linked hyaluronic acid has the effect of increasing the rigidity of the obtained nanometric fibers, but also of increasing the complexity of the three-dimensional structure of a film obtained by continuous deposition on several layers of the obtained fibers. In particular, the presence of cross-linked hyaluronic acid causes the formation of cavities in the film, cavities that allow the molecules of the active ingredient to be housed.

According to another example, the active ingredient is a non-steroidal anti-inflammatory, to be applied for example on a skin burn. It may also be provided to add, as additional active ingredients one or more analgesics, to soothe the pain caused by the burn. For this type of application, it is particularly advantageous that the first compound is of the skin regenerating type, such as hyaluronic acid.

The use of the composition according to the invention in the treatment of skin burns is beneficial because it results in a rapid absorption of the active ingredient and also of the first compound in the wound. Furthermore, the product obtainable by electrospinning the composition may be applied directly onto the burnt area. This improves the efficacy of the treatment.

Advantageously, the active ingredient is present at a concentration of 0.1% to 30% by weight, more preferably 0.2% to 20% by weight, even more preferably 0.5% to 10% by weight.

Note that hyaluronic acid, as a compound to be electrospun, is a good candidate to be combined with different active ingredients, of each of the three types indicated above.

For example, among the cosmetic active ingredients can be mentioned those that are anti-seborrhoeic (sebacic acid, azelaic acid), antioxidants (ascorbic acid, tocopherol, retinol, retinal), anti-blemish (glabridin, ammonium persulfate), emollients (Hamamelis virginiana extract, bisabolol) and humectants (e.g., glycerin, propanediol)

Among the preferred nutritional active ingredients are natural products manufactured using intact sources or substances extracted or derived from plants, animals, algae, fungi, lichens or bacteria (phytosterol, echinacea, green tea extract, garlic, aloe vera, fish oil, spirulina, chlorella, digestive enzymes derived from fungi), vitamins (e.g., vitamins A, B, C, D, E, K, folic acid, biotin) and antioxidants (for example lipoic acid, coenzyme Q10, flavonoids, glutathione, resveratrol, catechins).

The most advantageous pharmaceutical active substances are androgens and anabolic steroids (for example testosterone), anti-CTLA-4 monoclonal antibodies (e.g., ipilimumab), anti-PD-1 monoclonal antibodies (e.g., nivolumab), anti-anginal agents (e.g., nitroglycerin), anti-asthmatic combinations (e.g., diphylline/guaifenesin), antibiotics (e.g., metronidazole), antibiotics/antineoplastics (e.g., doxorubicin), antineoplastics (e.g., isotretinoin), and antineoplastic combinations (e.g., letrozole/ribociclib).

It is to be noted that such active ingredients can be advantageously combined with other compounds to be electrospun such as, for example, xanthan gum, guar gum, chondroitin sulfate, collagen or starch.

Another example of the composition is heparin as a compound to be electrospun and a pharmaceutical active ingredient, for example, an allergen extract or a platelet stimulating agent such as eltrombopag.

According to embodiments, the composition also comprises a stabilizer, which is preferably a cross-linkable polymer. An example of a stabilizer is sodium alginate, which should be added to pullulan as a promoter. Preferably the pullulan: alginate ratio is from 3:1.5 to 3:0.5, more preferably 3:1.

The present application is also intended to protect a method for preparing the composition described so far, said process providing to mix together the components of the composition in water or in an aqueous solution. It can be provided that all the components are mixed together at the same time or are added one after another. It may also be provided to solubilize each ingredient separately in a respective aliquot of water, and then to mix the aliquots together so as to obtain the composition, or one or two components in an aliquot.

The surfactant is preferably added some time before electrospinning is performed, as this allows the electrospinning to be more stable, in the interests of a more regular and defect-free electrospun fiber.

Electrospinning of the above composition, by the above method, leads to the realization of one or more electrospun fibers, in particular nanofibers, which form a membrane substrate. Such a membrane substrate may be in the form of a non-woven fabric.

The fibers produced are substantially composed of the compounds of the composition described above. In particular, the fibers are composed of the first compound to be electrospun, the electrospinning promoter and the surfactant. If provided for in the composition, the fibers also include the active ingredient and/or the stabilizer.

With particular regard to the active ingredient, it is noted that it can be integrated into the fibers, resulting from an electrospinning of the active ingredient together with the rest of the composition, or it can be separated from the fibers but inserted into the membrane substrate.

It has been observed that the electrospun fiber can have a flat ribbon shape, that is, it has a cross-section of flat or flattened shape.

### EXAMPLES

Electrospinning tests of the following composition were performed with industrial equipment:

| Compound | Concentration (wt %) | Quantity (g) |
|---|---|---|
| Water | 78.84 | 236.5 |
| Pullulan | 10 | 30 |
| Hyaluronic acid | 10 | 30 |
| NaOH | 0.33 | 1 |
| Biosurfactin | 0.83 | 2.5 |
| TOTAL | 100 | 300 |

The composition was prepared by dissolving the hyaluronic acid in 100 g of water, dissolving the pullulan in 100 g of water and dissolving the surfactin and base in 36.5 g of water.

The three aliquots of water with the respective components were mixed together, and the obtained solution was loaded into equipment of an industrial type. It was possible to produce an A4 size fiber layer in 2 hours. It has been observed that the fiber obtained is regular and free of defects, and has a substantially ribbon shape, i.e., it has a flat or flattened section.

It is clear that modifications and/or additions of parts or steps may be made to the composition and to the method for preparation as described heretofore, without departing from the field and scope of the present invention as defined by the claims.

It is also clear that, although the present invention has been described with reference to some specific examples, a person of skill in the art shall certainly be able to achieve other equivalent forms of a composition to be electrospun, having the characteristics as set forth in the claims and hence all coming within the field of protection defined thereby.

1. A composition to be electrospun, comprising a first compound to be electrospun selected from xanthan gum, pectins, chitin, chitosan, dextran, carrageenan, guar gum, agar, cellulose derivatives, albumin, starch, gelatin, collagen, elastin, μ-glucans, glycosaminoglycans, mucopolysaccharides, water-soluble polysaccharides and their derivatives; and an electrospinning promoter selected from alginate, pullulan and a mixture thereof; wherein the componsition also comprises a surfactant of biological and organic origin obtained by fermentation of gram positive microorganisms.

2. The composition as in claim 1, wherein said surfactant is a lipopeptide obtained by Bacillus sp.

3. The composition as in claim 2, wherein said surfactant is selected from Surfactins, Pumilacidins and Lichenysins.

4. The composition as in claim 1, wherein said surfactant is concentrated at 0.1-5% by weight of the total weight of said composition.

5. The composition as in claim 1, wherein the composition has a pH comprised between 6 and 7.

6. The composition as in claim 5, wherein the composition comprises a basic compound at a concentration lower than 2% by weight of the total weight of said composition.

7. The composition as in claim 1, wherein the weight ratio between said first compound to be electrospun and said electrospinning promoter is comprised between 2:1 and 1:2.

8. A method to prepare a composition to be electrospun, comprising a step of mixing a first compound to be electrospun with an electrospinning promoter and a surfactant, wherein said first compound to be electrospun is selected from xanthan gum, pectins, chitin, chitosan, dextran, carrageenan, guar gum, agar, cellulose derivatives, albumin, starch, gelatin, collagen, elastin, μ-glucans, glycosaminoglycans, mucopolysaccharides, water-soluble polysaccharides and their derivatives, said electrospinning promoter is selected from alginate, pullulan and a mixture thereof, and said surfactant is of biological and organic origin obtained by fermentation of gram positive microorganisms.

9. The method as in claim 8, wherein said surfactant is a lipopeptide obtained by Bacillus sp.

10. The method as in claim 9, wherein said surfactant is selected from Surfactins, Pumilacidins and Lichenysins.

11. The method as in claim 8, wherein the method provides to mix said first compound to be electrospun, said promoter and said surfactant in respective separate aliquots of water and subsequently to combine said aliquots of water.

12. The method as in claim 8, wherein the method provides to mix, together with said first compound to be electrospun, said electrospinning promoter and said surfactant, a basic compound so as to maintain the pH of the mixture between 6 and 7.

13. A patch product, comprising a membrane substrate absorbable by the skin, wherein said membrane substrate is formed by at least one electrospun fiber based on a first compound to be electrospun, an electrospinning promoter and a surfactant, wherein said first compound to be electrospun is selected from xanthan gum, pectins, chitin, chitosan, dextran, carrageenan, guar gum, agar, cellulose derivatives, albumin, starch, gelatin, collagen, elastin, μ-glucans, glycosaminoglycans, mucopolysaccharides, water-soluble polysaccharides and their derivatives, said electrospinning promoter is selected from alginate, pullulan and a mixture thereof, and said surfactant is of biological and organic origin obtained by fermentation of gram positive microorganisms.

14. The patch product as in claim 13, wherein said at least one electrospun fiber has the shape of a flat ribbon.

15. The patch product as in claim 13, wherein said surfactant is a lipopeptide obtained by Bacillus sp.

16. The patch product as in claim 15, wherein said surfactant is selected from Surfactins, Pumilacidins and Lichenysins.

17. The composition as in claim 2, wherein said surfactant is concentrated at 0.1-5% by weight of the total weight of said composition.

18. The composition as in claim 2, wherein the composition has a pH comprised between 6 and 7.

19. The method as in claim 9, wherein the method provides to mix said first compound to be electrospun, said promoter and said surfactant in respective separate aliquots of water and subsequently to combine said aliquots of water.

20. The method as in claim 9, wherein the method provides to mix, together with said first compound to be electrospun, said electrospinning promoter and said surfactant, a basic compound so as to maintain the pH of the mixture between 6 and 7.
